(19) United States
(12) Patent Application Publication
(10) Pub. No.: US 2004/0049122 A1
(43) Pub. Date: Mar. 11, 2004

(54) Oral thermometer probe covers

(76) Inventors: Sherin Lussier, North Providence, RI (US); Melinda Penney, Providence, RI (US); Dalita R. Tomellini, Rehoboth, MA (US)

Correspondence Address: FISH & RICHARDSON PC, 225 FRANKLIN ST, BOSTON, MA, 02110, US

(21) Appl. No.: 10/241,277

(22) Filed: Sep. 11, 2002

(51) Int. Cl. A61B 5/00

(52) U.S. Cl. 600/549

(57) ABSTRACT

Probe covers for oral digital thermometers are provided. The probe covers include a flavoring. Methods of making and using such probe covers are also provided.

### TECHNICAL FIELD

This invention relates to oral thermometer probe covers.

### BACKGROUND

Digital medical thermometers for measuring an individual's body temperature are commonly available for use both in clinical settings and in the home. Clinical digital thermometers typically include an elongated metal probe that is attached by a coil cable to an electronic base. Home digital thermometers are generally small, battery-operated units with a relatively short probe. Both types of thermometers may be specifically adapted for oral use.

Disposable thermometer probe covers are used in both hospital and home settings to prevent the spread of infection and disease from one individual to another. For example, in the clinical setting the use of a disposable probe cover facilitates the availability of a sanitary probe and probe cover for use on each new patient while eliminating the necessity for sterilizing the probe after each use.

Probe covers for digital thermometers for home use are commercially available, e.g., from CVS, Inc. or Valu-Rite. As shown in FIGS. 1-3, such probe covers typically include a sleeve 10 that is generally flat prior to insertion of the digital thermometer 11. Sleeve 10 is formed by sealing two plastic layers 12, 14 together along an edge having a shape that generally corresponds to the outline of the digital thermometer 16 (FIG. 1). The plastic sleeve is provided on a paper backing 16, to which it is releasably bonded by a pressure-sensitive adhesive (not shown). The paper backing is typically generally rectangular, as shown in FIG. 2.

Clinical probe covers are commercially available, e.g., from Becton Dickinson Company. Such probe covers may include a thermoplastic tube, e.g., a tube of rigid high density polyethylene, into which the elongated probe is inserted. An example of this type of probe cover is shown in FIG. 4. Probe cover 30 includes a tapered cylindrical tube 32 that is configured to fit the elongated probe of a standard clinical digital thermometer, and a rim 34.

### SUMMARY

The inventors have found that improved aesthetic characteristics, and appeal to the individual whose temperature is being taken, can be provided by imparting a flavor to probe covers for oral digital thermometers. The addition of a flavor will tend to make temperature-taking more acceptable to individuals who find the procedure unpleasant, particularly children.

In some cases, the probe cover will also include a fragrance, which may be combined with the flavor if desired.

The invention also features methods of manufacturing probe covers and methods of taking the temperature of a human patient.

Other features and advantages of the invention will be apparent from the description and drawings, and from the claims.

### DESCRIPTION OF DRAWINGS

FIG. 1 is a schematic top view of an oral digital thermometer for home use, with a probe cover attached.

FIG. 2 is a schematic top view of the probe cover of FIG. 1 prior to use.

FIG. 3 is a schematic side view indicating the layers of a laminate suitable for use in the probe cover of FIG. 1.

FIG. 4 is a side view of a probe cover suitable for use with an oral digital thermometer for clinical use.

### DETAILED DESCRIPTION

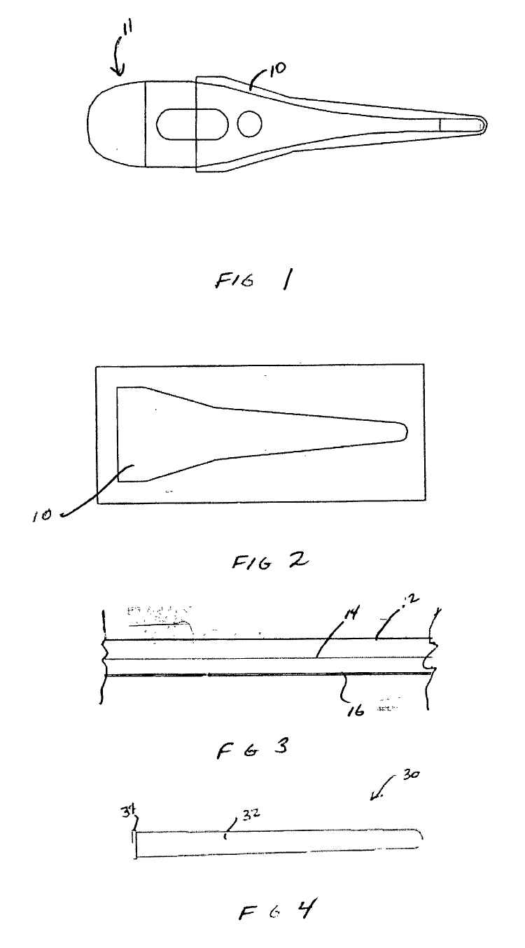

Suitable probe covers include those described above with reference to FIGS. 1-4.

The flavoring may include any flavoring substance suitable for use in the mouth, e.g., flavorings used in food products and in other products that come into contact with the oral cavity such as dental flosses and toothpastes. Suitable flavorings include flavor oils, e.g., peppermint, wintergreen, cinnamon and the like.

Flavor oils are relatively volatile, and thus in some cases could be lost during storage of the product. If desired, the flavor may be provided in the form of spray-dried particles, for enhanced stability. Spray-dried flavor particles may be contained on the surface of or partially embedded in a water-insoluble wax coating. The spray-dried flavor particles consist of the flavor oil dispersed in a water-soluble matrix material. Suitable water-soluble materials include gums such as gum acacia, gum arabic, gum tragacanth and the like; starches such as corn starch; dextrins and the like. Suitable materials for use as flavors are those which allow the user to detect a strong, noticeable flavor while permitting the maintenance of an acceptable product appearance. Such flavors include peppermint, spearmint, wintergreen, cassia, cinnamon and the like; fruit flavors such as cherry, strawberry, lime; and the like.

The spray-dried flavor particles can be readily prepared by known spray drying procedures. For example, a suitable flavor oil can be mixed with a solution of a water-soluble matrix material and the resulting mixture is then emulsified by mechanical or other means to form an emulsion. The emulsion thus formed is then passed through suitable spray-drying apparatus to flash off the water present in the emulsion resulting in the formation of a spray-dried flavor particle. The spray-dried flavor particles should be of a particle size of from about 44.mu. to 840 μm with particles of 177 μm or smaller being preferred. As discussed above, the spray-dried flavor particles consist of a flavor dispersed in the matrix of a water-soluble medium. The flavor comprises from about 15 to 25% by weight of the spray-dried flavor particle and the water-soluble medium comprises from about 85-75% by weight of the spray-dried flavor particle.

The flavor oil may also be provided in a solution that facilitates application of the flavor oil to the probe cover, and adhesion and stability of the flavor oil during storage of the product.

The flavoring may be applied to the probe cover using any suitable technique. For example, the flavoring may be applied by known coating methods.

In the case of the home use probe covers shown in FIGS. 1-3, the flavoring may be applied to either or both of the plastic film layers 12, 14 (FIG. 3). Alternatively, the flavoring may be applied to the pressure sensitive adhesive that adheres the probe cover to the paper backing 16 or to the paper backing layer 16, in which case the flavoring will be absorbed by the plastic layer 14 (or both layers 12, 14) during storage.

The flavoring may be applied during manufacture of the film and/or paper layers, or may be subsequently applied, e.g., by spraying, brushing, roll coating, or other suitable methods. The flavoring may also be incorporated into the pressure sensitive adhesive during its manufacture or after it has been applied to the paper backing.

In the case of the clinical use probe covers (FIG. 4), the flavoring may be applied to part of the outer surface of the cover, or the entire area if desired. If desired, the flavoring may be applied as a strip, or to only one side of the cover, or to only a portion of the length of the cover. The flavoring may be applied by any suitable technique, including dipping, spraying, or brushing. Preferably, after application the flavoring is in the form of a dry powder-like film. The flavoring may be applied in the form of a powder, provided sufficient adhesion to the plastic is attained, or as a liquid.

To further enhance the aesthetic appeal of the product, the plastic of the home or clinical probe covers, and/or the paper backing of the home covers, may be colored and/or provided with graphics, e.g., the probe cover may be printed with a cartoon character or other graphic that would be appealing to children.

A number of embodiments of the invention have been described. Nevertheless, it will be understood that various modifications may be made without departing from the spirit and scope of the invention.

For example, while certain probe cover designs are shown in FIGS. 1-4, the invention may be practiced with probe covers having other designs. Moreover, the flavoring may be provided in any desired form, for example as a solution, suspension, powder, or in microencapsulated form. In addition, the paper backing described above may be replaced by a polymeric film or other type of backing if desired.

Accordingly, other embodiments are within the scope of the following claims.

What is claimed is:

1. A probe cover comprising
a body configured to fit over the probe of an oral digital thermometer and allow body temperature to be measured by the thermometer through the body; and
a flavoring disposed on an outer surface of the body.

2. A probe cover of claim 1 wherein the body comprises a flexible sleeve.

3. A probe cover of claim 2 wherein the sleeve comprises opposed sheets of plastic film sealed at their edges.

4. A prove cover of claim 1 wherein the body comprises a rigid or semi-rigid sleeve.

5. A probe cover of claim 4 wherein the sleeve is generally cylindrical and is tapered to conform to the shape of a probe of a clinical digital thermometer.

6. A probe cover of claim 4 wherein the sleeve comprises high-density polyethylene.

7. A probe cover of claim 1 further comprising a fragrance disposed on a surface of the body.

8. A probe cover of claim 7 wherein the fragrance and flavoring are combined.

9. A probe cover of claim 1 wherein the flavoring comprises a flavor oil.

10. A probe cover of claim 1 wherein the body includes a graphic.

11. A probe cover of claim 1 wherein the body is formed of a colored plastic.

12. A method of manufacturing a probe cover comprising:
(a) providing a body configured to fit over the probe of an oral digital thermometer and allow body temperature to be measured by the thermometer through the body; and
(b) applying a flavoring to a surface of the body.

13. A method of claim 12 wherein the applying step comprises spraying the flavoring onto the body.

14. A method of claim 12 wherein the applying step comprises dipping the body in a solution comprising the flavoring.

15. A method of claim 12 wherein the body is generally flat prior to use, and the applying step comprises rolling the flavoring onto the body.

16. A method of claim 12 wherein the flavoring is provided in a carrier that enhances adhesion of the flavoring to the body.

17. A method of claim 12 further comprising providing the body on a backing sheet, wherein the applying step comprises applying the flavoring to the backing and contacting the surface with the backing.

18. A method of taking the temperature of a human, comprising:
(a) providing a digital oral thermometer having a probe portion;
(b) covering the probe portion with a probe cover comprising a body configured to receive the probe portion and a flavoring disposed on a surface of the body; and
(c) placing the probe portion and probe cover in the oral cavity of the human.
